(19) United States
(12) Patent Application Publication
(10) Pub. No.: US 2014/0212515 A1
(43) Pub. Date: Jul. 31, 2014

(54) COMPOSITION AND METHODS FOR OPTIMIZING HEALTH OF CANCER PATIENTS BY THE ADMINISTRATION OF SODIUM SELENITE AND VITAMIN K3

(71) Applicant: Mark Ira Simon, Westlake Village, CA (US)

(72) Inventor: Mark Ira Simon, Westlake Village, CA (US)

(21) Appl. No.: 13/755,219

(22) Filed: Jan. 31, 2013

(51) Int. Cl. A61K 33/04; A61K 45/06; A61K 31/198; A61K 31/122

(52) U.S. Cl. CPC A61K 33/04; A61K 31/122; A61K 45/06; A61K 31/198
USPC 424/702

(57) ABSTRACT

Compositions and methods of treating carcinoma in an individual afflicted with such a condition, comprising the administration to the individual a therapeutically effective amount of a composition consisting essentially of a nutraceutically acceptable carrier and sodium selenite, with or without menadione. The administration may be sublingual and may be performed in conjunction with injection, into the solid tumor where the carcinoma is a solid tumor, or by intra-arterial catheterization or an organ specific artery where the carcinoma is organ specific, or both.

### CROSS-REFERENCE TO RELATED APPLICATIONS

Not Applicable.

### STATEMENT REGARDING FEDERALLY SPONSORED RESEARCH OR DEVELOPMENT

Not Applicable

### NAMES OF PARTIES TO A JOINT RESEARCH AGREEMENT

Not Applicable

### REFERENCE TO SEQUENCE LISTING, A TABLE, OR A COMPUTER PROGRAM LISTING COMPACT DISK APPENDIX

Not Applicable

### FIELD OF INVENTION

The invention relates to an anti-neoplastic nutraceutical composition consisting essentially of a nutraceutically acceptable carrier and a therapeutically effective amount of one sodium selenite, with or without menadione. The admistration is sublingual, and may be accompanied by injection into a solid tumor mass, intra-arterial injection into an organ specific artery, or both.

### BACKGROUND OF INVENTION

The use of nutritional supplements, including selenium, in preventing or treating carcinoma is well-known in the art and sublingual and injectable formulations or other nutritional supplements are known. For instance, U.S. Pat. No. 5,639,787, to Riordan, et al, discloses a method of treating cancer by intravenous infusion, an anti-cancer effective dose of ascorbic acid or a pharmaceutically acceptable salt thereof.

Selenium is a a metalloid believed to be antioxidant due to its presence in the active centers of Reactive Oxygen Species, decomposing peroxidases and superoxide dismutases. Selenium exists in soil mainly as a mineral sodium selenite in a wide spectrum of concentrations; thus its content in food products depends very much on a geographical area they are grown. Amongst organic forms of selenium the most abundant is Selenocysteine is most abundant of organic forms and is now recognized as the 21st amino acid being present in about fourteen mammalian selenoproteins. However, only sodium selenite and not the organic forms of selenium reacts rapidly with sulfhydryl groups (SH) of proteins (P) oxidizing them to disulfides. Lipinski, B, 2011 Oxidative Medicine and Cellular Longevity 1-9 (2011). Selenium, vitamin E, and isoflavonoid supplementation has been used in prostate cancer patients and a decrease in Prostate

Specific Antigen was noted in 64%. Anagnostu, T, et al, 7(1) Rev Uro1.18-40 (2005). Superoxide anion generated by selenite triggers mitochondrial damage and subsequent mitophagy, leading to irreversible cell death in glioma cells. Kim, et al, 67(13) Cancer Res 6314-24 (2007). Mitochondrial induction of apoptosis has also been shown by Eric Olm, Cytotoxic Mechanisms of Selenium in Cancer, Karolinska Institutet, Stockholm 2009. In vitro studies of mesothelioma lines showed increased apoptosis with selenite exposure, Nilsonne, G, 28 Journal of Experimental & Clinical Cancer Research 92 (2009) and studies of prostate cancer cells showed an induction of P53, Sarveswaran, S, 36 Int Jl Oncology 1419-1428 (2010). The use of high dose sodium selenite as a chemotherapy adjuvant resulted in a statistically significant increase in percentage of reduction of cervical and axillary lymphadenopathy, decrease in splenic size, and decreased percentage of bone marrow infiltration in lymphoma patients. Asfour, I, et al, 127 Biol Trace Elem Res 127:200-210 (2009).

Fatty Acid Synthase (FASN) is not only overexpressed in cancer, but it also plays an essential role in tumour growth and survival. Two well-known FASN inhibitors, the natural product cerulenin and the synthetic compound c75, have been studied. Treating tumour cells with pharmacological inhibitors of FASN effectively suppresses growth and induces apoptosis in breast cancer cells both in vitro and in vivo, suggesting that FASN inhibitors could show selective and enhanced cytotoxicity to cancer cells under the tumour microenvironment conditions. T Mashima, H Seimiyal and T Tsuruo. 100 British Journal of Cancer 1369-1372 (2009). Other authors have inhibition of FASN as an alternative to selective estrogen receptor modulators, Lupu, R, and Menendez, J, 147(9) Endocrinology 4056-4066 (2006).

Menadione, Vitamin K3, has been reported to inhibit the proliferation of rat pancreatic cancer cells, Osada, S, et al, 28 Anticancer Research 45-50 (2008). Similar results are reported in human prostate cancer cell lines for restriction of the amino acid Methionine, Lu, S, and Epner, D, 38(1) Nutrition and Cancer 123-130 (2000) and its use in human cancer suggested. Payne, A., 1 Med Hypothesis Research 247-252(2004). The restriction of the amino acid arginine in cancer treatment has also been suggested. Wheatley, D, 1(1) Oncology News 6-9 (2006).

The invention teaches an anti-neoplastic composition consisting of sodium selenite, with or without menadione, for in the treatment of carcinoma that may be administered as a sublingual and requires none of the additives previously used for sublingual administration such as hemostatic agents, polymers, carrier particles. U.S. Pat. No. 6,939,559, to Nishibi, et al, discloses a pharmaceutical composition for sublingual administration comprising, along with a medicament, one or more hemostatic agents, one or more water-insoluble and/or low water soluble substance, having an osmotic pressure of 72 mOsm or less. U.S. Pat. No., 6,183,775, to Ventouras, discloses a controlled release lozenge consisting of at least 50 weight percent of a soluble filler of maltitol, xylitol, sorbitol, mannitol, lactose, dextrose, saccharose, fructose, and mixtures thereof; and 0.5 to 30 weight percent each of an insoluble film forming agent polyacrylate, ethyl cellulose, polyvinylchloride, cellulose acetate, cellulose acetate phthalate, shellac, and a swellable polymer which is selected from the group consisting of xanthan gum, guar gum, alginic acid or a salt thereof, pectin, polyvinyl alcohol, polysaccharide, cellulose derivatives, and mixtures thereof along with the active substance. U.S. Pat. No. 6,761,910, to Pettersson, discloses an essentially water-free, ordered mixture of microparticles of at least one pharmaceutically active agent adhered to the surfaces of carrier particles, said particles being substantially larger than said microparticles and being water-soluble, and a bioadhesion and/or mucoadhesion promoting agent mainly adhered to the surfaces of the carrier particles.

### BRIEF DESCRIPTION OF INVENTION

This inventor has discovered an anti-neoplastic nutraceutical composition consisting essentially of a nutraceutically acceptable carrier and a therapeutically effective amount of one sodium selenite to be therapeutically beneficial and in some cases curative, for carcinoma. The nutraceutically acceptable carrier may be comprised of one or more binders, excipients, buffers, and flavorants and the flavorants may be selected from calcium carbonate, dextrose, sodium carbonate, magnesium stearate, mannitol, sorbitol, and xylitol, all of which have been discovered to be suitable for sublingual, intravenous injection, and intra-arterial catheterization.

Over 30 years of scientific evidence supports the use of sodium selenite(Na2SeO3) as a selective cytotoxic agent for the prevention and treatment of most forms of cancer. Recent clinical studies have demonstrated that high dose sodium selenite is safe and effective as a therapy for a wide variety of cancers. Sodium selenite triggers apoptosis through a mitochondrial mediated pathway, mediated by the generation of Radical Oxygen Species (ROS) that exceed the capacity of antioxidant systems to control redox balance. There are numerous secondary mechanisms including immunological responses and reactivation p53 transcription. Neoplastic cells have a high affinity for selenite and lack the catalase enzyme that neutralizes hydrogen peroxide while healthy cells are unaffected by sodium selenite within the dose range that is cytotoxic for neoplastic cells. A method for administration of high dose sodium selenite has been developed that reduces risk of negative side effects and toxicity. This is a very low-cost alternative to conventional chemotherapy and can be administered at home with oral sublingual administration.

Vitamin K3 (Menadione, 2-Methyl-1,4-Napthoquinone), a synthetic version of naturally occurring vitamins K1 and K2, has shown anti-tumor activity when used alone. High dose sodium ascorbate therapy is greatly enhanced by the co-administration of Menadione. Menadione, when administered alone or with sodium ascorbate, increases redox cycling which sustains the pro-oxidant effects of ascorbate. The combination of sodium selenite and Menadione similarly enhances the oxidation of free radicals.

### DETAILED DESCRIPTION OF INVENTION

In the following description, numerous specific details are set forth in order to provide a more thorough description of the present invention. It will be apparent, however, to one skilled in the art, that the present invention may be practiced without these specific details. In other instances, well-known features have not been described in detail so as not to obscure the invention.

In the Summary above, the Description of the Invention, and the Claims and Abstract below, reference may be made to particular features (including method steps) of the invention. It is to be understood that this disclosure includes possible combinations of such particular features.

For example, where a particular feature is disclosed in the context of a particular aspect or embodiment of the invention, or a particular claim, that feature may also be used, to the extent possible, in combination with and/or in the context of other particular aspects and embodiments of the invention, and in the invention generally.

The term “comprises” and grammatical equivalents thereof are used herein to mean that other components, ingredients, steps etc. are optionally present. For example, an article “comprising” (or “which comprises”) components A, B and C can consist of (i.e. contain only) components A, B and C, or can contain not only components A, B and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number or the indefinite article “a” (meaning “one”) is used herein to denote the start of a range beginning with that number (which may be a range having an upper limit or no upper limit, depending on the variable being defined). For example “at least one” or “at least a” means 1 or more than 1. The term “at most” followed by a number is used herein to denote the end of a range ending with that number (which may be a range having 1 or 0 as its lower limit or a range having no lower limit, depending upon the variable being defined). For example, “at most 4” means 4 or less than 4, and “at most 40%” means 40% or less than 40%. If, in this disclosure, a range is given as “(a first number) to (a second number)” or “(a first number)−(a second number)”, this means a range whose lower limit is the first number and whose upper limit is the second number. For example, 0-10 mm means a range whose lower limit is 0 mm, and whose upper limit is 10 mm.

The term “or” is used herein as a conjunction used to link alternatives in a series of alternatives. The term “and/or” is used herein as a conjunction meaning that either or both of two options may be valid.

This inventor has discovered an anti-neoplastic nutraceutical composition consisting essentially of a nutraceutically acceptable carrier and a therapeutically effective amount of one sodium selenite. The nutraceutically acceptable carrier may be comprised of one or more binders, excipients, buffers, and flavorants and the flavorants may be selected from calcium carbonate, dextrose, sodium carbonate, magnesium stearate, mannitol, sorbitol, and xylitol. In a specific embodiment, the sodium selenite is present in an amount of about 500 mcg-50 mg per tablet, and more specifically the sodium selenite is present in an amount of about 1.25 mg to about 10 mg per tablet. The binders, excipients, buffers, and flavorants and the flavorants may be selected from calcium carbonate, dextrose, sodium carbonate, magnesium stearate, mannitol, sorbitol, and xylitol, all of which are suitable for sublingual administration.

In a second embodiment, the anti-neoplastic nutraceutical composition consists of a nutraceutically acceptable carrier and therapeutically effective amounts of sodium selenite and menadione. The nutraceutically acceptable carrier may be comprised of one or more binders, excipients, buffers, and flavorants; and the binders, excipients and flavorants may be selected from the of calcium carbonate, dextrose, sodium carbonate, magnesium stearate, and stevia. More specifically, the sodium selenite is present in an amount ranging from about 500 mcg-50 mg per tablet and the menaquinone is present in an amount ranging from about 1 mg to about 500 mg per tablet; and most specifically the sodium selenite is present in an amount of about 1.25 mg to about 10 mg and the menadione is present in an amount ranging from about 10 mg to about 50 mg per tablet.

Also disclosed is a method of treating carcinoma in an individual afflicted with such a condition, comprising the administration to the individual a therapeutically effective amount of a composition consisting essentially of a nutraceutically acceptable carrier and sodium selenite. The administration is, most specifically, sublingual and the nutraceutically acceptable carrier comprises one or more binders, excipients, buffers, and flavorants. More specifically, the administration is sublingual and the sodium selenite is present in an amount of about 500 mcg to about 50 mg per tablet and the and performed one to four times per day; most specifically the sodium selenite is present in an amount of about 1.25 mg to about 10 mg and the administration is sublingual and is performed two times per day. The method may be further combined with one or more additional anti-neoplastic therapies such as chemotherapy, radiation, FASN inhibitors, selective amino acid restriction. Where amino acid restriction is used, the amino acid to be restricted is, most specifically, methionine.

In a related method of treating carcinoma in an individual afflicted with such a condition, the individual is administered a therapeutically effective amount of a composition consisting essentially of a nutraceutically acceptable carrier and sodium selenite and menadione.The administration is, most specifically, sublingual and the nutraceutically acceptable carrier comprises one or more binders, excipients, buffers, and flavorants that may be selected from the of calcium carbonate, dextrose, sodium carbonate, magnesium stearate, and stevia. More specifically, the sodium selenite is present in an amount ranging from about 500 mcg-50 mg per tablet and the menaquinone is present in an amount ranging from about 1 mg to about 500 mg per tablet and the administration is performed between one and four times per day; and most specifically the sodium selenite is present in an amount of about 1.25 mg to about 10 mg and the menadione is present in an amount ranging from about 10 mg to about 50 mg per tablet and the administration is performed two times per day. In this embodiment, the method may be further combined with one or more additional anti-neoplastic therapies such as chemotherapy, radiation, FASN inhibitors, selective amino acid restriction. Where amino acid restriction is used, the amino acid to be restricted is, most specifically, methionine.

In a third embodiment of the invention, the carcinoma is a solid tumor and the administration to the individual a therapeutically effective amount of a composition consisting essentially of a nutraceutically acceptable carrier and sodium selenite, with or without menadione, is performed in conjunction with the direct injection of sodium selenite into a solid tumor mass, more specifically at a dose of about 25 mcg to about 300 mcg per cubic centimeter of tumor volume as a solution at a concentration of about 300 mcg to about 3 mg of sodium selenite per milliliter of H2O. Most specifically, the direct injection of sodium selenite is at a dose of about 100 mcg per cubic centimeter of tumor volume as a solution at a concentration of about 1 mg of sodium selenite per millileter of H2O. The injection may be performed at multiple locations within the tumor, under local anesthetic, utilizing a needle and syringe, said syringe being compressed as said needle is withdrawn.

In yet a third embodiment, the method of treating carcinoma in an individual afflicted with such a condition, comprising the sublingual administration to the individual of a therapeutically effective amount of a composition consisting essentially of a nutraceutically acceptable carrier and sodium selenite, with or without menadione, is performed in conjunction with intra-arterial catheterization of an organ specific artery of a solution at a concentration of about 300 mcg to about 3 mg per milliliter of H2O, with or without injection of the solution directly into the solid tumor mass. More specifically, the solution is at a concentration of about 1 mg of sodium selenite per milliliter of H2O and most specifically, the organ is the liver and the organ specific artery is the hepatic artery or a branch of the hepatic artery.

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize or be able to ascertain using no more than routine experimentation, many equivalents to the specific embodiments of the invention described specifically herein. Such equivalents are intended to be encompassed in the scope of the claims.

What is claimed is:

1. An anti-neoplastic nutraceutical composition consisting essentially of a nutraceutically acceptable carrier and a therapeutically effective amount of one sodium selenite.

2. The anti-neoplastic nutraceutical composition of claim 1 wherein the nutraceutically acceptable carrier comprises one or more binders, excipients, buffers, and flavorants.

3. The anti-neoplastic nutraceutical composition of claim 2 wherein the binders, excipients and flavorants are selected from the group consisting of calcium carbonate, dextrose, sodium carbonate, magnesium stearate, mannitol, sorbitol, and xylitol.

4. The anti-neoplastic nutraceutical composition of claim 1 wherein the sodium selenite is present in an amount of about 500 mcg-50 mg per tablet.

5. The anti-neoplastic nutraceutical composition of claim 4 wherein the sodium selenite is present in an amount of about 1.25 mg to about 10 mg per tablet.

6. An anti-neoplastic nutraceutical composition consisting of a nutraceutically acceptable carrier and therapeutically effective amounts of sodium selenite and menadione.

7. The anti-neoplastic nutraceutical composition of claim 6 wherein the nutraceutically acceptable carrier comprises one or more binders, excipients, buffers, and flavorants.

8. The anti-neoplastic nutraceutical composition of claim 7 wherein the binders, excipients and flavorants are selected from the of calcium carbonate, dextrose, sodium carbonate, magnesium stearate, and stevia.

9. The anti-neoplastic nutraceutical composition of claim 9 wherein the sodium selenite is present in an amount ranging from about 500 mcg-50 mg per tablet and the menaquinone is present in an amount ranging from about 1 mg to about 500 mg per tablet.

10. The anti-neoplastic nutraceutical composition of claim 9 wherein the sodium selenite is present in an amount of about 1.25 mg to about 10 mg and the menadione is present in an amount ranging from about 10 mg to about 50 mg per tablet.

11. A method of treating carcinoma in an individual afflicted with such a condition, comprising the administration to the individual a therapeutically effective amount of a composition consisting essentially of a nutraceutically acceptable carrier and sodium selenite.

12. The method of claim 11, wherein the administration is sublingual the nutraceutically acceptable carrier comprises one or more binders, excipients, buffers, and flavorants.

13. The method of claim 12 wherein the sodium selenite is present in an amount of about 500 mcg to about 50 mg per tablet and the administration is sublingual and performed one to four times per day.

14. The method of claim 13 wherein the sodium selenite is present in an amount of about 1.25 mg to about 10 mg and the administration is sublingual and is performed two times per day.

15. The method of claim 14 further comprising one or more anti-neoplastic therapies selected from the group consisting of chemotherapy, radiation, FASN inhibitors, selective amino acid restriction.

16. The method of claim 15 wherein the therapy is selective amino acid restriction and the amino acid is methionine.

17. A method of treating carcinoma in an individual afflicted with such a condition, comprising the administration to the individual a therapeutically effective amount of a composition consisting essentially of a nutraceutically acceptable carrier and sodium selenite and menadione.

18. The method of claim 17 wherein the administration is sublingual and the nutraceutically acceptable carrier comprises one or more binders, excipients and flavorants.

19. The method of claim 18 wherein the sodium selenite is present in an amount ranging from about 500 mcg-50 mg per tablet and the menadione is present in an amount ranging from about 1 mg to about 500 mg per tablet and the administration is performed between one and four times per day.

20. The method of claim 19 wherein the sodium selenite is present in an amount of about 1.25 mg to about 10 mg, the menadione is present in an amount ranging from about 10 mg to about 50 mg per tablet and the is performed two times per day.

21. The method of claim 20 further comprising one or more anti-neoplastic therapies selected from the group consisting of chemotherapy, radiation, FASN inhibitors, selective amino acid restriction.

22. The method of claim 21 wherein the therapy is selective amino acid restriction and the amino acid is methionine.

23. The method of claim 13 wherein the carcinoma is a solid tumor and the method further comprises the direct injection of sodium selenite into the solid tumor at a dose of about 25 mcg to about 300 mcg per cubic centimeter of tumor volume as a solution at a concentration of about 300 mcg to about 3 mg of sodium selenite per milliliter of H2O.

24. The method of claim 23 wherein said direct injection of sodium selenite is at a dose of about 100 mcg per cubic centimeter of tumor volume as a solution at a concentration of about 1 mg of sodium selenite per millileter of H2O.

25. The method of claim 24 wherein said injection is performed at multiple locations within the tumor, under local anesthetic, utilizing a needle and syringe, said syringe being compressed as said needle is withdrawn.

26. The method of claim 13 wherein said carcinoma is organ specific and the method further comprised intra-arterial catheterization of an organ specific artery and the injection of a solution at a concentration of about 300 mcg to about 3 mg per milliliter of H2O.

27. The method of claim 26 wherein said solution is at a concentration of about 1 mg of sodium selenite per milliliter of H2O.

28. The method of claim 27 wherein said organ specific carcinoma is of the liver and said organ specific artery is the hepatic artery or a branch of the hepatic artery.
